(19) United States
(12) Patent Application Publication
(10) Pub. No.: US 2026/0130576 A1
(43) Pub. Date: May 14, 2026

(54) INDEPENDENTLY MOVABLE EXTERNAL CHANNEL FOR A MEDICAL DEVICE, AND ASSOCIATED SYSTEMS AND METHODS

(71) Applicant: Boston Scientific Scimed, Inc., Maple Grove, MN (US)

(72) Inventors: Alexander R. Cohen, Austin, TX (US); Ryan Davis Gilkey, Austin, TX (US); Timothy Noah Foster-Frau, Austin, TX (US)

(73) Assignee: Boston Scientific Scimed, Inc., Maple Grove, MN (US)

(21) Appl. No.: 19/382,615

(22) Filed: Nov. 7, 2025

Related U.S. Application Data
(60) Provisional application No. 63/718,207, filed on Nov. 8, 2024.

(51) Int. Cl. A61B 1/00

(52) U.S. Cl. CPC A61B 1/00098

(57) ABSTRACT

A device, system, and method providing a channel external or in addition to a working channel of a medical device such as a medical scope. The external channel is separately and independently movable with respect to the working channel of the medical device. A first medical instrument may thus be delivered through the working channel of the medical device to a treatment site within a patient’s body, and a second medical instrument may be separately delivered through the external channel and to the treatment site. The first instrument may be manipulated to perform a first aspect of the procedure with respect to the treatment site, and the second instrument may be operated and manipulated independently of the first instrument to perform another, second aspect of the procedure independently of the first aspect.

### CROSS-REFERENCE TO RELATED APPLICATIONS

This application claims the benefit of priority under 35 U.S.C. § 119 to U.S. Provisional Application No. 63/718,207, filed November 8, 2024, the entire disclosure of which is hereby incorporated by reference herein for all purposes.

### FIELD

The present disclosure relates generally to the field of medical devices deliverable and usable within a patient’s body. More particularly, the present disclosure relates to devices, systems, and methods providing an external medical device along a medical device, and movable independently of the medical device, such as within a patient’s body.

### BACKGROUND

Minimally-invasive procedures, such as transluminal, transcatheter, percutaneous, endoscopic, etc., procedures, have various benefits over open surgery. Whereas open surgery requires cutting open the patient’s body to gain access into the patient’s body and internal anatomical structures, minimally-invasive procedures access a target site within the patient via a natural orifice (or, in some instances, a small incision not considered to constitute an open-surgery cut). Such methods induce little to no bleeding (minimized to no surgical cutting), require minimal if any implements to hold open the access opening, reduce various risks typical of open surgery, reduce recovery times, and have various other benefits over open surgery. Despite the various benefits over open surgery, the limited working space within a patient available for performing minimally-invasive procedures presents various challenges. Often more than one medical instrument, tool, device, etc., is used during a given medical procedure. However, the lumen of a delivery device for such medical instruments, tools, devices, etc., often does not accommodate more than one medical instrument, tool, device, etc., to be extended therethrough. Therefore, multiple device exchanges may be required during the procedure. Moreover, the maneuverability of the instruments at the target site for the procedure may be limited by the delivery device through which the instrument extends. During Endo Luminal Surgery (ESL) procedures, such as endoscopic surgical procedures, there may be a need to add additional channels to a medical scope (e.g., an endoscope) to deploy multiple tools. However, maneuverability and control of the multiple channels and or devices may present a challenge, particularly if different movements and/or actions are to be performed by the different devices. For instance, during Endoscopic Submucosal Dissection (ESD), a physician may want to apply tissue traction to help visualize the separation being performed between the mucosa / submucosa and the muscle / muscularis. In this instance, there generally is a need to deploy a tool or device to apply traction, and then make that tool or device independent of the movement directions of the endoscope (through which a tissue-separating device is extended). However, typical deployment of a traction device through the working channel of the endoscope limits the movement and operation of the traction device to being affected by, and not independent of, movements of the endoscope. As may be appreciated, the limited spaces in which minimally-invasive procedures typically are performed also impacts the maneuverability of various accessories, such as traction devices, within the body. Accordingly, there remains a need for improved devices, systems, and associated methods to deliver and use a medical device independently of another medical device, such as a medical scope, within a patient.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a system for performing a procedure within a patient’s body includes a first elongate member configured to deliver a medical device for use during the procedure and extending along a longitudinal axis, the first elongate member having a distal end with a distalmost end, a proximal end, and an exterior extending between the distal end and the proximal end; a second elongate member configured to deliver a medical device for use during the procedure and extending along and coupled to the first elongate member, the second elongate member having a distal end with a distalmost end, and a proximal end, and an exterior extending between the distal end and the proximal end; a movable member positioned between the first elongate member and the second elongate member and operable to move the distal end of the second elongate member laterally away from the distal end of the first elongate member; and a return element coupling the first elongate member and the second elongate member, and movable to move the second elongate member laterally toward the first elongate member.

In some aspects, the second elongate member is coupled to the first elongate member along the distal ends of the second elongate member and the first elongate member proximal to the distalmost ends of the second elongate member and the first elongate member. In some aspects, the system further includes an external coupler coupling the exterior of the second elongate member and the exterior of the first elongate member.

In some aspects, the first elongate member defines an interior lumen therethrough, and the return element is coupled to the distal end of the second elongate member and extends proximally through the interior lumen defined through the first elongate member. In some aspects, the return element extends proximally to a control handle accessible outside the patient.

In some aspects, the movable member is an expandable member. In some aspects, the expandable member extends circumferentially around the exterior of the second elongate member. In some aspects, the system further comprises an inflation lumen extending along the second elongate member to be in fluid communication with the expandable member and an inflation medium source.

In some aspects, the first elongate member, the second elongate member, or both of the first elongate member and the second elongate member define at least one lumen therethrough.

In some aspects, the first elongate member is a flexible tubular elongate member of a medical scope; the flexible tubular elongate member is configured to be inserted into the patient, defines a working channel therethrough, and has an increased-flexibility section extending from the distalmost end of the first elongate member proximally to a transition point along the distal end of the first elongate member; and the second elongate member is coupled to the first elongate member at or along the transition point.

In accordance with various principles of the present disclosure, a device is configured to deliver a medical instrument to a treatment site within a patient alongside another medical device. In some aspects, the device includes an elongate member having a distal end with a distalmost end, and a proximal end, and an exterior extending between the distal end and the proximal end; a movable member mounted on the elongate member and operable to move the distal end of the second elongate member laterally away from the other medical device; and a return element coupled to the distal end of the elongate member and capable of being coupled to the other medical device, the return element extending proximally to a control handle accessible outside the patient and configured to move the elongate member closer to the other medical device.

In some aspects, the movable member is an expandable member.

In some aspects, the movable member is an inflatable member extending circumferentially around the exterior of the elongate member.

In some aspects, the device further comprises an external coupler configured to couple the exterior of the elongate member to the exterior of the other medical device.

In some aspects, the elongate member defines a lumen extending from the proximal end of the elongate member to the distal end of the elongate member, the lumen configured for delivery of a medical instrument therethrough.

In accordance with various principles of the present disclosure, a method is disclosed for delivering more than one medical instrument to a treatment site within a patient’s body. In some aspects, the method includes inserting a distal end of a first elongate member and a distal end of a second elongate member into a patient's body; advancing distalmost ends of the first elongate member and the second elongate member to the treatment site; moving the distal end of the second elongate member laterally away from the distal end of the first elongate member; and operating a return element to return the distal end of the second elongate member laterally toward the first elongate member.

In some aspects, the method further includes coupling the distal end of the second elongate member to the distal end of the first elongate member at or proximal to a transition point between an increased flexibility section of the first elongate member extending from the distalmost end of the first elongate member proximally to the transition point and a proximally extending section of the first elongate member extending proximally from the transition point.

In some aspects, the return element is coupled to the distal end of the second elongate member and extends through a working channel defined through the first elongate member, the method comprising proximally pulling on the return element to return the distal end of the second elongate member laterally toward the first elongate member.

In some aspects, the method further includes expanding an expandable member to move the distal end of the second elongate member laterally away from the distal end of the first elongate member.

In some aspects, the method further includes inflating an inflatable member, circumferentially extending around the exterior of the second elongate member, to move the distal end of the second elongate member laterally away from the distal end of the first elongate member. These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of a system for performing procedures within a patient’s body and formed in accordance with aspects of the present disclosure.

FIG. 2 illustrates an elevational view of a system such as illustrated in FIG. 1 positioned and in use with respect to a schematic representation of treatment site within a patient.

FIG. 3 illustrates a view similar to that of FIG. 2, but at a further stage of use of the illustrated system.

FIG. 4 illustrates a cross-sectional view along line IV-IV of FIG. 3.

FIG. 5 illustrates a perspective view of a system such as illustrated in FIG. 3, illustrating relative lateral movement between an endoscope and an auxiliary device formed in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

The present disclosure describes various medical devices and systems which may be delivered into a patient’s body via minimally invasive techniques. Minimally invasive procedures are becoming more desirable for removing or repairing target tissue within or in the area of a target site within a patient’s body. In contrast with open surgery, minimally invasive surgery involves advancing a medical device into a patient’s body through a natural orifice (or small incision, if necessary) and navigated the medical device within the body, such as through an internal body passage, to a target site. It will be appreciated that terms such as devices, instruments, tools, components, accessories, etc., may be used interchangeably herein without intent to limit unless otherwise indicated. It will further be appreciated that terms such as target site, target area, target tissue site, target tissue area, target area of tissue, target treatment area, treatment area, target treatment site, treatment site, etc., may be used interchangeably herein, without intent to limit, to refer to an area or region of tissue within which a target tissue is located. The term target tissue (and other variations thereof, such as treatment tissue) is used herein to refer to the tissue with respect to which a procedure is to be performed or which is to be treated or otherwise operated on or affected by the devices and/or systems and/or methods disclosed herein. Thus, the target site may be understood as an area or region extending outwardly from or around or surrounding the target tissue (specific tissue in the target tissue area), such as the region a medical professional would consider a working area around the target tissue for performing a procedure with respect to the target tissue.

Typically, an elongate member is used in minimally-invasive medical procedures to deliver a medical device to a target site. Typically, the elongate member is sufficiently flexible to navigate through curved and/or tortuous body passages, particularly if inserted transluminally. In some aspects, the elongate member is tubular, such as in the form of a catheter, sheath, tube, cannula, etc. (such terms being used interchangeably herein without intent to limit), and defines a lumen through which a medical device is advanced to a target site. A tubular elongate member may be used as a delivery device, introducer, etc. A tubular elongate member may or may not have a medical device mounted or otherwise coupled to a distal end thereof. In some aspects, it may be advantageous to deliver a medical device through a delivery device in the form of a tubular elongate member to protect or shield the passage through which the medical device is delivered (e.g., body passage or otherwise) against adverse interactions (e.g., catching, scratching, and/or otherwise) by the medical device delivered therethrough. Additional overtubes or sheaths may be provided as desired or as necessary to reduce friction or interference of the exterior of any elements with another element.

In addition to the above-described elongate members, various medical devices, such as medical scopes (e.g., endoscopes, arthroscopes, bronchoscopes, colonoscopes, cystoscopes, duodenoscopes, gastroscopes, hysteroscopes, laparoscopes, ureteroscopes, etc.), include an elongate member for insertion into a body passageway or cavity to enable a medical professional to deliver a medical device to a target site within a patient’s body to perform minimally-invasive surgical procedures at the target site internal to the patient's body. Medical scopes typically have a proximal end (typically with a control handle) that remains external to the patient, and a distally extending flexible elongate member configured for insertion into the patient. The distally-extending flexible elongate member is generally tubular, and may be known as an insertion tube (and may referenced herein as such for the sake of convenience and without intent to limit). The distally-extending flexible elongate member of the medical scope is inserted into a body cavity or passage of the patient, and is navigated within the patient to an internal treatment site. In general, the distally-extending flexible elongate member of the medical scope has one or more working channels extending longitudinally therethrough through which a medical device may be advanced or retracted. The distally-extending flexible elongate member of the medical scope may also be equipped with one or more accessories such as, for example, a miniature viewing device (optical component, such as a camera), an illumination device (e.g., an LED or optical fiber), lumens for suction, lumens for inflation / irrigation mediums, etc. The distally-extending flexible elongate members of most medical scopes generally have a limited number of working channels, and the outer diameter of such devices generally is limited by the size of the body passage through which the device is to be navigated.

For purposes of the present disclosure, an elongate member with a medical device at a distal end thereof, or a tubular elongate member through which another medical device may be delivered to a target site, or the distally-extending flexible elongate member of a medical scope, or another form of an elongate member may be considered herein to be a delivery device, or, more generally an “elongate member” with which systems, devices, and methods of the present disclosure may be used. For the sake of convenience, reference is made herein simply to an elongate member. It should be appreciated that such elongate member may have a medical device mounted or coupled to a distal end thereof (e.g., as an integral device), or may be a tubular elongate member with a medical device extended therethrough. The elongate member may be any suitable size, cross-sectional shape or area, and/or configuration permitting introduction and passage of devices or instruments to the distal end of the elongate member, such as for passage and introduction of medical instruments to a target tissue site. It will be appreciated that the elongate member need not define a lumen therethrough (need not be tubular), but, instead, may include a shaft with a medical device mounted thereon (e.g., on the distal end thereof). Typically, the elongate member has at least a distal portion which is elongate and flexible to be able to be navigated through passages within a patient’s body which may be curved and/or tortuous, with a proximal portion extending proximally (out of the patient’s body or coupled with a control handle outside the patient’s body) to be controlled (maneuvered, or one or more functions thereof, such as camera, irrigation, suction, light, etc., actuated) by a medical professional. An auxiliary delivery system formed in accordance with various principles of the present disclosure may be delivered with (e.g., over) such an elongate member to a target site.

Various elongate members may be delivered to a treatment site within a patient’s body for delivery and use of a tool for performing a procedure with respect to the treatment site. In accordance with various principles of the present disclosure, an additional elongate member is provided in addition to a primary elongate member. The additional elongate member optionally provides one or more channels, in addition to a channel which may be provided by the primary elongate member, to deliver one or more additional devices within a patient’s body, or may provide one or more additional devices without an additional channel. In some aspects, the additional elongate member may be considered to be auxiliary to the primary elongate member, and may be alternately referenced herein as an auxiliary elongate member without intent to limit. In some aspects, it is desirable for the auxiliary elongate member to be movable and/or otherwise operable independently of the primary movement directions and/or operation of the primary elongate member.

The auxiliary elongate member may have a significantly smaller outer diameter than the elongate member so as not to interfere with advancing of the elongate member within a patient’s body to a target site. The auxiliary elongate member may extend along the exterior of the primary elongate member and may thus alternately be referenced as an external elongate member. It will be appreciated that the addition of an external elongate member along the exterior of the primary elongate member in accordance with various principles of the present disclosure increases the overall diameter and cross-sectional area of the system less than if the primary elongate member were modified to include another working channel therethrough. Moreover, the addition of another working channel to the primary elongate member might impact the flexibility of the primary elongate member as it is navigated within the body (such as through tortuous body passages), whereas an auxiliary elongate member providing an external channel having a lumen dimensioned for a filament or a single working instrument may be generally more flexible than the primary elongate member. Moreover, the external elongate member does not impact the flexibility of an existing elongate member because the external elongate member need not be permanently coupled with the primary elongate member along the entire length of the primary elongate member, and may be coupled with the primary elongate member along only a limited extent (e.g., only along the distal end of the primary elongate member) for delivery with the primary elongate member, as discussed in further detail below.

It will be appreciated that an external channel formed in accordance with various principles of the present disclosure may be particularly beneficial / useful in conjunction with a tubular elongate member which only has a single delivery lumen (e.g., an endoscope with a single working channel), or a lumen of limited diameter. As may be appreciated, the external channel allows for delivery of additional / auxiliary medical devices while a medical device is delivered through the working channel of the tubular elongate member. In some aspects, the external elongate member may be considered a “sidecar” to the primary elongate member, providing a channel or tool external to and alongside the primary elongate member In accordance with various principles of the present disclosure, the external elongate member is movable independently of the primary elongate member, and therefore may allow the additional medical device to move independently of the primary elongate member, its working channel, and/or any medical device delivered by the primary elongate member. As may be appreciated, auxiliary elongate member formed in accordance with various principles of the present disclosure thereby provide greater flexibility to the medical professional in the use of medical devices and the operation of such devices with respect to tissue at a target site.

In accordance with various principles of the present disclosure, the auxiliary elongate member is coupled with respect to the primary elongate member to be advanced with the primary elongate member into a patient’s body and to a treatment site. In some aspects, the auxiliary elongate member is operably coupled with the primary elongate member in any of a variety of manners including, without limitation, mechanically coupling (e.g., taping, tying, banding, etc.) adhering (e.g., with adhesive), bonding, welding, etc., the present disclosure not being limited in this regard. In accordance with various principles of the present disclosure, the auxiliary elongate member is coupled along a distal end of a primary elongate member at a location spaced proximally from the distalmost end of the primary elongate member. In some aspects, the primary elongate member has a distal increased flexibility section extending proximally from the distalmost end thereof to a transition point. The flexibility of the primary elongate member may be relatively lower distal to the transition point than proximal to the transition point. Nonetheless, it will be appreciated that decreased flexibility of the primary elongate member proximal to the transition point may still be sufficient to facilitate navigation of the primary elongate member through curved (e.g., tortuous) body passages. The increased flexibility section of the primary elongate member may be more flexible to allow more controlled movement of the increased flexibility section, such as to direct the distalmost end toward a selected location (e.g., to direct a visualization element, light, or working channel of the primary elongate member to such location). In accordance with various principles of the present disclosure, the auxiliary elongate member is coupled at or proximal to the transition point along the primary elongate member so as to be movable independently of the movement of the increased flexibility section of the primary elongate member. In some aspects, the auxiliary elongate member may be pivotable about the point of attachment to the primary elongate member. As may be appreciated, the auxiliary elongate member optionally has an increased flexibility section along the distal end thereof, substantially similar in nature to the increased flexibility section of the primary elongate member. Because the auxiliary elongate member is not coupled to the primary elongate member from a distalmost end of the auxiliary elongate member to the attachment point, the increased-flexibility section of the primary elongate member may flex and/or be moved laterally with respect to the auxiliary elongate member. In some aspects, the auxiliary elongate member may roll or otherwise move laterally with respect to the longitudinal axis of the primary elongate member.

In some aspects, it may be desirable to return the auxiliary elongate member to a position adjacent the primary elongate member, such as to return the distalmost end of the auxiliary elongate member to adjacent the distalmost end of the primary elongate member. In accordance with various principles of the present disclosure, a control member may operably associate or couple the distal end of the auxiliary elongate member with the primary elongate member. In some aspects, the control member is operable to maintain a distal section of the auxiliary elongate member adjacent to the primary elongate member, or to allow the distal section of the auxiliary elongate member to move independently of the primary elongate member. For instance, the control member may be released or slackened to allow the auxiliary elongate member to be moved away from the primary elongate member. The auxiliary elongate member may thus track the distal end of the primary elongate member, such as the curvature of the distal end of the primary elongate member, as desired or needed (e.g., during delivery or withdrawal), yet may be decoupled from the primary elongate member as desired to move independently of the primary elongate member (e.g., during a procedure). The control member may be used to return the distal section of the auxiliary elongate member to a position adjacent to the primary elongate member. For instance, movement of the auxiliary elongate member independent of the primary elongate member may be desired for a limited time during a procedure, whereas during other times it may be desirable to move the auxiliary elongate member in conjunction with the primary elongate member, and/or at least to have the distal section of the auxiliary elongate member adjacent to the primary elongate member. The control member allows for selectable and adjustable positioning of the auxiliary elongate member with respect to the primary elongate member as desired or medically indicated during a procedure.

One specific case (without limiting the scope of the present disclosure) in which an auxiliary elongate member such as formed in accordance with various principles of the present disclosure may be used is in aiding in Endoscopic Submucosal Dissection (ESD). In ESD, a physician may want to apply tissue traction to help visualize the separation between the mucosa / submucosa and the muscle / muscularis. In this instance, there is a need to deploy a tool or device, and then make that tool or device independent of the movement directions of the endoscope. This allows the physician to complete the dissection using the scope articulation without the traction vector changing with every scope movement. This also allows for potential general improvements in tissue manipulation like laparoscopic tools used today.

Various embodiments of external medical channels and/or elongate members, and associated devices, systems, and methods, will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified by common reference elements in the drawings and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element).

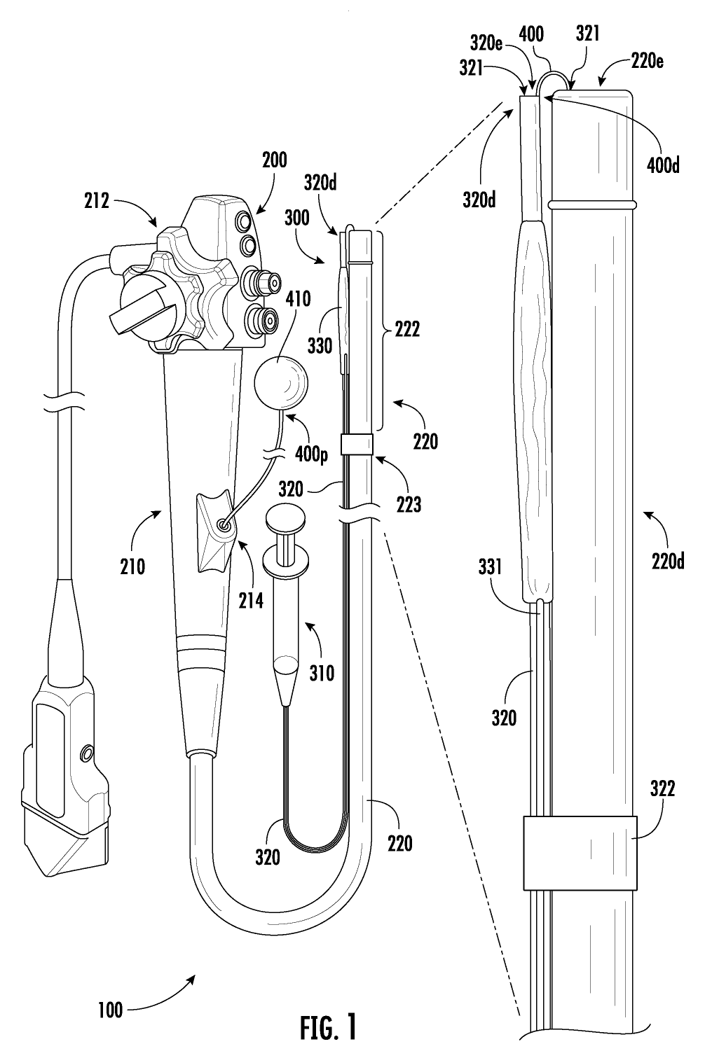

Turning now to the drawings, an example of an embodiment of a system 100 formed in accordance with various principles of the present disclosure for performing procedures within a patient’s body is illustrated in FIG. 1. The system includes a medical scope 200 (e.g., an endoscope) and an auxiliary device 300 extending along the medical scope 200. In some aspects, the auxiliary device 300 may be considered a “sidecar” to the medical scope 200, providing functionality and/or delivering another medical instrument usable in conjunction with the medical scope 200. It will be appreciated that other suitable configurations of a medical scope 200 may be utilized than the medical scope 200 illustrated in FIG. 1 and discussed herein.

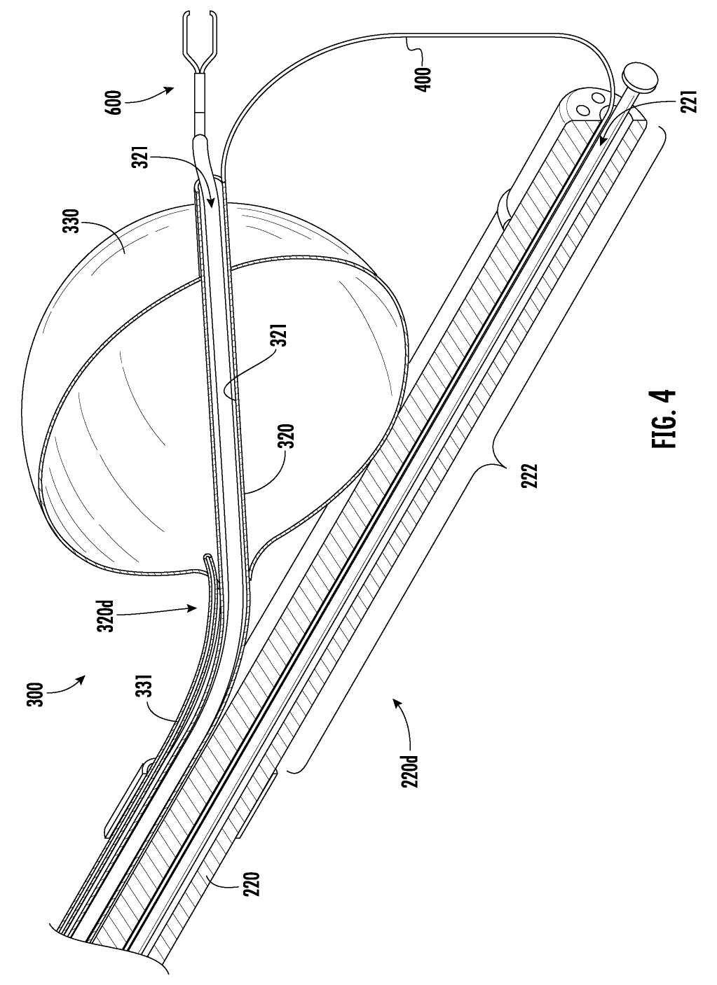

The illustrated example of a medical scope 200 includes a control handle 210 along a proximal end 200p thereof, and a flexible tubular elongate member 220 (also known as a tubular shaft or insertion member of the medical scope) extending distally from the control handle 210. The flexible tubular elongate member 220 has a distal end 220d which is insertable into a patient to be advanced to a target area within a patient’s body in a manner known to those of ordinary skill in the art. The flexible tubular elongate member 220 defines one or more lumens or working channels 221 (see, e.g., FIG. 4) through which one or more medical tools may be extended and delivered to the target area.

The auxiliary device 300 defines an external elongate member 320 which extends along the flexible tubular elongate member 220 of the medical scope 200. The external elongate member 320 may be a shaft of a medical instrument and configured to deliver the medical instrument (such as alongside the flexible tubular elongate member 220) to a treatment site within a patient’s body. In some aspects, the external elongate member 320 is an external channel defining a lumen 321 therethrough. An external elongate member 320 in the form of an external channel may be considered to define an additional or auxiliary lumen for use with the medical scope 200. For instance, a medical instrument in addition to a medical instrument delivered by (e.g., through the working channel 221 of) the medical scope 200 may be delivered through the lumen 321 of the external elongate member 320 to a treatment site.

In some aspects, the external elongate member 320 is coupled to the flexible tubular elongate member 220 for insertion with the flexible tubular elongate member 220 into the patient’s body. For instance, it may be desirable or necessary for the external elongate member 320 (e.g., the distal end 320d of the external elongate member 320) to follow the flexible tubular elongate member 220 (e.g., the distal end 220d of the flexible tubular elongate member 220) to the treatment site. In some aspects, the external elongate member 320 may be coupled at a specific circumferential orientation with respect to the flexible tubular elongate member 220, such as with respect to an element of the distalmost end 220e of the flexible tubular elongate member 220, such as the outlet of the working channel 221 of the flexible tubular elongate member 220 and/or a port (e.g., for inflation, suction, irrigation, etc.) and/or a visualization element (e.g., camera, fiber optic, etc.) and/or an illumination or lighting element, such as commonly provided through a flexible tubular elongate member 220 of a medical scope, as known to those of ordinary skill in the art. Typically, the position of the external elongate member 320 along the circumference of the flexible tubular elongate member 220 is set prior to insertion of the flexible tubular elongate member 220 and the external elongate member 320 into the patient’s body.

The distal end 220d of the flexible tubular elongate member 220 of the medical scope 200 typically has an increased-flexibility section 222 more flexible than the portion of the flexible tubular elongate member 220 proximal to the increased-flexibility section 222. The increased-flexibility section 222 may be considered as the portion of the flexible tubular elongate member 220 distal to a transition point 223, and the proximal portions of the flexible tubular elongate member 220 which are not as flexible as the increased-flexibility section 222 may be considered to be proximal to the transition point 223. The control handle 210 may include one or more control knobs 212 for controlling movement of the increased-flexibility section 222 of the flexible tubular elongate member 220, such as to direct the distalmost end 220e of the flexible tubular elongate member 220 toward a selected location (e.g., to direct a visualization element, light, or working channel of the elongate member to such location), in a manner known to those of ordinary skill in the art. For instance, small movements of the distalmost end 220e of the flexible tubular elongate member 220 may be needed to control movement of an instrument extended through the working channel 221 and out the distalmost end 220e of the flexible tubular elongate member 220.

In accordance with various principles of the present disclosure, the external elongate member 320 is coupled to the flexible tubular elongate member 220 at a position proximal to the distalmost end 320e of the external elongate member 320 and proximal to the distalmost end 220e of the flexible tubular elongate member 220. In some aspects, the external elongate member 320 is coupled to the flexible tubular elongate member 220 at or proximal to the transition point 223 between the increased-flexibility section 222 and the remaining proximal extent of the flexible tubular elongate member 220 proximal to the increased-flexibility section 222. Coupling the external elongate member 320 at such location along the distal end 220d of the flexible tubular elongate member 220 allows for maximization of relative movement between the distal end 320d of the external elongate member 320 and the increased-flexibility section 222 of the flexible tubular elongate member 220. In the illustrated example of an embodiment, an external coupler 322, such as, without limitation, a band, strap, tie, etc., may be used to couple the external elongate member 320 with the flexible tubular elongate member 220. However, the present disclosure need not be limited in this regard. In some aspects, the external coupler 322 allows axial translation of the external elongate member 320 with respect to the flexible tubular elongate member 220 of the medical scope 200 without lateral separation therefrom. In some aspects, the external coupler 322 is removable to allow separation of the external elongate member 320 from the flexible tubular elongate member 220. Coupling the external elongate member 320 to the flexible tubular elongate member 220 at or proximal to the transition point 223 allows for the full extent of movement (e.g., flexion) of the increased-flexibility section 222 of the flexible tubular elongate member 220, as well as movement of the distal end 320d of the external elongate member 320 relative to the increased-flexibility section 222.

The external elongate member 320 may be moved with respect to the flexible tubular elongate member 220 in any of a variety of manners. For instance, a movable member operable to move the external elongate member 320 with respect to the flexible tubular elongate member 220 may be positioned to be operably associated with the external elongate member 320 and the flexible tubular elongate member 220. In some aspects, the movable member is mounted on one or both of the external elongate member 320 or the flexible tubular elongate member 220. In the example of an embodiment illustrated in FIG. 1, a movable member in the form of an expandable member 330 is provided between the wall defining the external elongate member 320 and the wall defining the flexible tubular elongate member 220. However, other elements may be used to move the external elongate member 320 without departing from the principles of the present disclosure. In some aspects, the expandable member 330 is inflatable, such as an expandable balloon. In some aspects, the expandable member 330 is mounted on the external elongate member 320. In some aspects, the expandable member 330 is formed circumferentially around the external elongate member 320, such as annular shaped, such as illustrated in FIG. 1. However, other configurations, such as limited circumferentially to adjacent surfaces of the external elongate member 320 and the flexible tubular elongate member 220, are also within the scope of the present disclosure.

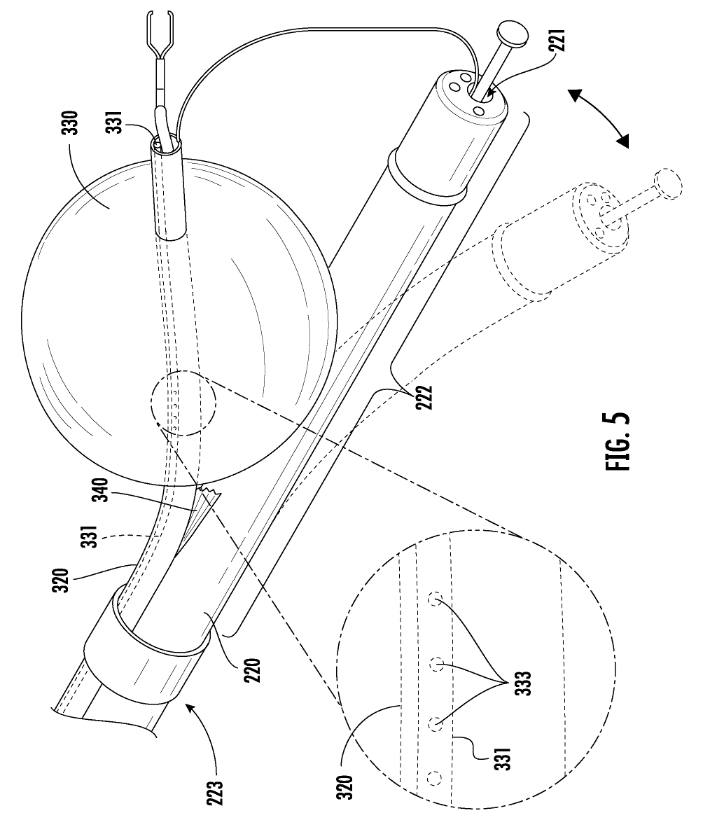

The interior of the example of an embodiment of a movable member in the form of an expandable member 330 is in fluid communication with a fluid source, such as provided by a control handle 310 of the auxiliary device 300. In some aspects, the control handle 310 is in the form of a syringe containing an inflation medium (e.g., a fluid such as saline, air, etc.). The control handle 310 is fluidly coupled with the interior of the expandable member 330 via an inflation lumen 331 to supply inflation medium to the expandable member 330. In the example of an embodiment illustrated in FIG. 1, the inflation lumen 331 is illustrated as provided defined by a tubular wall extending alongside the external elongate member 320, and in fluid communication with the inflation medium within the control handle 310 as well as with the interior of the expandable member 330. Alternatively, the inflation lumen 331 may be formed within the external elongate member 320 (such as alongside, but separate from, the lumen 321 defined through the external channel 320), and be fluidly communicated with the expandable member 330 via radially extending apertures 333 through the wall of the external elongate member 320 in a manner such as known to those of ordinary skill in the art, such as illustrated in FIG. 5. The control handle 310 may be adjusted or otherwise manipulated to control flow of the inflation medium to the expandable member 330 to control expansion /inflation, or collapse / deflation of the expandable member 330. As may be appreciated, expansion of the expandable member 330 moves the external elongate member 320 away from the flexible tubular elongate member 220, and deflation of the expandable member 330 allows the external elongate member 320 to be moved closer to the flexible tubular elongate member 220.

It will be appreciated that various properties, features, aspects, etc., of the expandable member 330 may be controlled, varied, selected, etc., to affect the movement of the external elongate member 320 with respect to the flexible tubular elongate member 220. For instance, the degree to which the expandable member 330 is expanded / inflated (e.g., the diameter of the expandable member 330) typically affects the angle of the external elongate member 320 with respect to the flexible tubular elongate member 220. In some aspects, such angle is defined by the tangency of the expandable member 330 with respect to the external elongate member 320 and/or the flexible tubular elongate member 220. Additionally or alternatively, such angle is defined by the flexibility of the external elongate member 320 and/or of the flexible tubular elongate member 220. and/or various other properties of the system 100. Various material properties of the expandable member 330 may also affect or influence the operation and effect of expansion of the expandable member 330. For instance, the expandable member 330 may be formed from a noncompliant material, such as polyethylene terephthalate (PET) or nylon, which may define a particular geometry in a fully expanded configuration. Alternatively, a compliant or semicompliant material, such as latex, silicone, etc., may allow greater variation in expansion configurations and/or variations of wall thicknesses which may impart different stiffness to the expandable member 330 along its geometry. Such properties may be selected based on various factors such as, without limitation, the procedure to be performed with the system 100, the needs of the medical professional, the location of the treatment site, etc.

In some instances, it may be desirable or medically indicated to move the distal end 320d of the external elongate member 320 away from the distal end 220d of the flexible tubular elongate member 220, and then, afterwards, to return the distal end 320d of the external elongate member 320 to a position closer to the distal end 220d of the flexible tubular elongate member 220. In accordance with various principles of the present disclosure, a control member is provided between the flexible tubular elongate member 220 and the external elongate member 320 and manipulable to control the relative positions of the external elongate member 320 and the flexible tubular elongate member 220, such as to move the external elongate member 320 closer to the flexible tubular elongate member 220. In the example of an embodiment illustrated in FIGS. 1-5, the control member is in the form of a return element 400 which may be an elongate flexible element, such as, without limitation, a filament, a string, a cord, a wire, a tether, etc. The return element 400 has a distal end 400d coupled (such as fixedly coupled) to the external elongate member 320, such as along the distal end 320d of the external elongate member 320. Additionally or alternatively, the distal end 400d of the return element 400 may be operably coupled with the expandable member 330. The proximal end 400p of the return element 400 extends outside the patient’s body for access and control by a medical professional. A grasping handle 410 (e.g., an enlarged grasping element) may be provided along the proximal end 400p of the return element 400 to facilitate grasping and/or manipulation of the return element 400. In the example of an embodiment illustrated in FIG. 1, the return element 400 is illustrated extending through a port 214 in the control handle 210 of the medical scope 200 to extend into the working channel 221 of the flexible tubular elongate member 220 of the medical scope 200. The return element 400 may extend from the proximal end 400p, through the working channel 221 of the flexible tubular elongate member 220, and out the outlet of the working channel 221 for coupling with the distal end 320d of the external elongate member 320. The medical professional may allow the return element 400 to be relatively slack to allow movement of the external elongate member 320 away from the flexible tubular elongate member 220 (e.g., during inflation of the expandable member 330). The return element 400 is translatable through the working channel 221 of the flexible tubular elongate member 220, and may be operated, such as pulled proximally, during or after deflation of the expandable member 330 to return the distal end 320d of the external elongate member 320 toward the distal end 220d of the flexible tubular elongate member 220. As may be appreciated, the return element 400 thus provides a return-to-home function which may not be reliably achievable by simply deflating the expandable member 330. It will be appreciated that such homing feature may be used more generally to adjust the distance between the distal end 320d of the external elongate member 320 and the distal end 220d of the flexible tubular elongate member 220, and is not limited to returning the distal end 320d of the external elongate member 320 to a position adjacent to (e.g., in contact with) the distal end 220d of the flexible tubular elongate member 220.

It will be appreciated that various modifications to the above-described examples may be made without departing from the principles of the present disclosure. For instance, as noted above, although the external elongate member 320 is described above as having a lumen 321 therethrough, principles of the present disclosure may be applied to a shaft of an instrument, the shaft not defining a lumen therethrough for passage of an instrument through the shaft. The shaft may nonetheless define an inflation lumen via which an expandable member may be inflated. The instrument may be configured to perform a procedure with respect to the treatment site (e.g., to engage, manipulate, operate on, such as cut, apply traction to, inject a medium into, etc., the tissue), or may be another type of device which does not interact with tissue, such as a camera, a light, an aspiration or irrigation source, etc. An expandable member 330 as described above may be advantageous in controlling movement of the shaft of the instrument, and thus the instrument, relative to tissue as well as relative to the medical scope 200. Additionally or alternatively, and as noted above, an auxiliary device 300 formed in accordance with various principles of the present disclosure may be used with a simple tubular delivery member (e.g., catheter, tubular shaft, etc.), instead of a medical scope 200, or with simply a shaft of an instrument, without a working channel or lumen defined through such shaft.

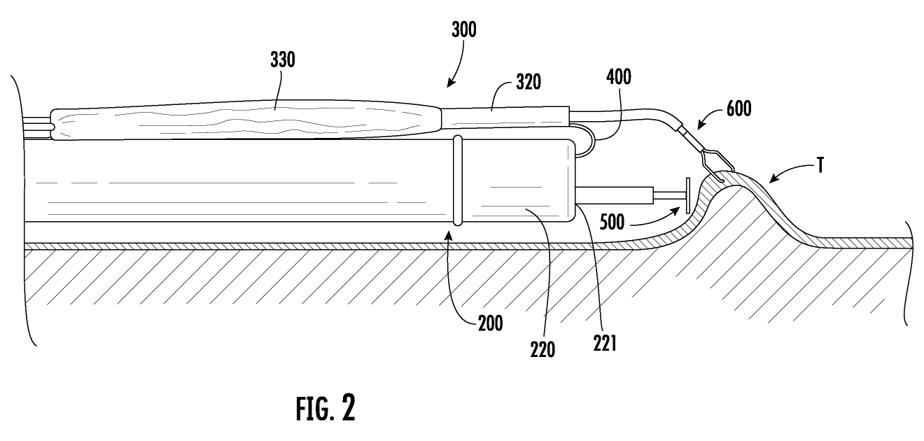

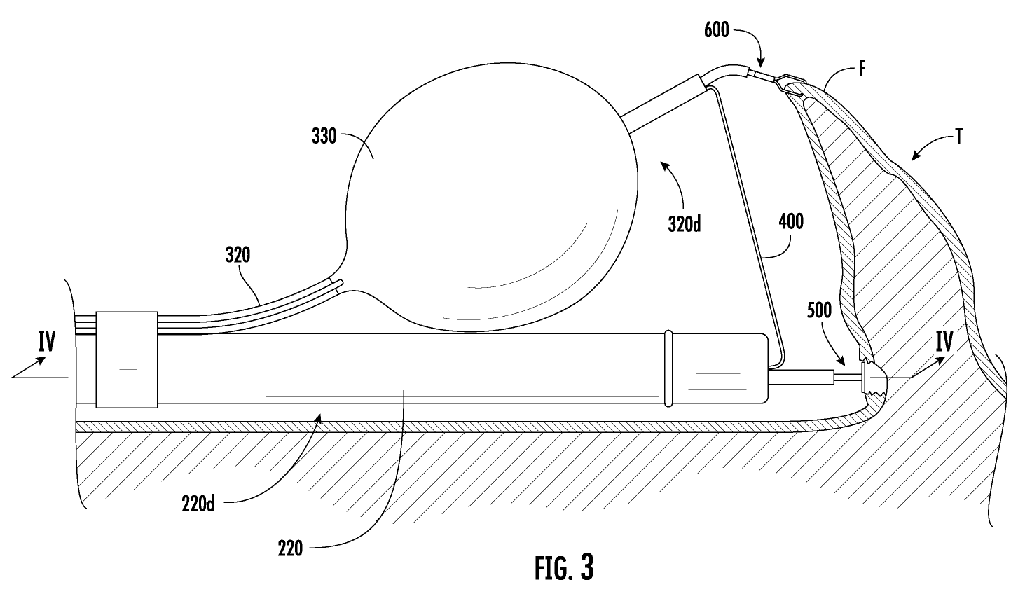

It will be appreciated that a system 100 formed in accordance with various principles of the present disclosure may be advantageously used in a variety of procedures. An example of a procedure with which the system 100 may be used, without limiting the scope of the present disclosure, is in aiding in Endoscopic Submucosal Dissection (ESD), such as schematically illustrated in FIG. 2, FIG. 3, and FIG. 5. As the tissue is being cut, a medical professional may want to apply traction to tissue above the cut / knife to help visualize the separation between the mucosa/ submucosa and the muscle / muscularis. In this instance, there is a need to deploy an auxiliary tool or device while another device is already deployed through the flexible tubular elongate member 220 of the medical scope 200. Furthermore, there is a need to move the auxiliary tool or device independently of the movement and use of tools or devices delivered through the flexible tubular elongate member 220 of the medical scope 200.

Using a system 100 formed in accordance with various principles of the present disclosure, a medical professional may complete the dissection using the medical scope articulation without the traction vector changing with every movement of the scope, such as schematically illustrated in FIG. 2, FIG. 3, FIG. 4, and FIG. 5. This also allows for potential general improvements in tissue manipulation like laparoscopic tools used today. In a tissue dissection procedure, the mucosal layer of tissue typically is lifted by injecting a lifting agent into the submucosal layer. For instance, saline may be injected to create a “bleb” of lifted tissue. As illustrated in FIG. 2, the flexible tubular elongate member 220 of a system 100 formed in accordance with various principles of the present disclosure may be used to deliver a cutting instrument 500 (blade, electrocautery knife, etc.) to the bleb at the target site T. The lifting agent which had been injected into the submucosal tissue may stretch the submucosal tissue layer to lift the mucosal tissue layer from the underlying muscularis tissue layer to protect the muscularis tissue layer from the cutting instrument 500. A traction device such as a tissue grasper 600 may be delivered through the auxiliary device 300 and used to assist with cutting the tissue at the target site T, such as by grasping and holding the tissue for cutting and/or holding the tissue taut. For instance, a the tissue grasper 600 may engage and move the tissue as needed as the cutting instrument 500 is used to continue to cut tissue. As may be appreciated, the system 100 allows for ready delivery of the cutting instrument 500 and the tissue grasper 600 to the target site S at the same time, without having to be extended through the same working channel 221 of the flexible tubular elongate member 220 of the medical scope 200. As noted above, the external elongate member 320 and the flexible tubular elongate member 220 may be coupled together for delivery together to the treatment site S. For instance, the external elongate member 320 may be coupled with respect to the flexible tubular elongate member 220 with the use of an external coupler 322 (such as illustrated, for example, in FIG. 1), and/or with the return element 400 held (e.g., pulled proximally) to maintain the distal end 320d of the external elongate member 320 generally adjacent to the distal end 220d of the flexible tubular elongate member 220, such as illustrated in FIG. 2.

Once the cutting instrument 500 cuts tissue at the treatment site S, a tissue flap F is created, as illustrated in FIG. 3. As also illustrated in FIG. 3, the tissue grasper 600 may be, e.g., simultaneously, used to grasp and lift the tissue flap F away from surrounding tissue and thus away from the cutting instrument 500 and/or a field of view of the medical scope 200. The expandable member 330 may be inflated, such as illustrated, to adjust the position of the external elongate member 320 with respect to the medical scope 200. As noted above, the inflation medium may be delivered to the expandable member 330 through an inflation lumen 331, such as illustrated in FIG. 4 (showing a cross-sectional view along line III-III of the system 100 as illustrated in FIG. 3). Additionally or alternatively, the return element 400 may be utilized to adjust the position of the external elongate member 320 of the auxiliary device 300 with respect to the flexible tubular elongate member 220 (e.g., specifically, the increased-flexibility section 222) of the medical scope 200.

As may be appreciated, the distal end 320d of the external channel 320 is decoupled from the distal end 220d of the medical scope 200, thereby allowing decoupled, independent movements of the cutting instrument 500 and the tissue grasper 600. The direction of the force vector applied to the tissue flap T by the tissue grasper 600 may thereby be adjusted independently of the cutting instrument 500 as the external elongate member 320 is moved independently of the medical scope 200. For instance, the increased-flexibility section 222 of the flexible tubular elongate member 220 of the medical scope 200 may be moved (e.g., left, right, up, or down) with respect to the treatment site S without affecting the position of the distal end 320d of the external elongate member 320 with respect to the tissue flap F. In some aspects, the expandable member 330 may roll, or otherwise move with respect to, the increased-flexibility section 222 as the increased-flexibility section 222 is moved (e.g., flexed or bent) independently of the expandable member 330, such as illustrated in FIG. 5. The rolling or other movement of the external elongate member 320 may dampen the affect the movements of the increased-flexibility section 222 of the flexible tubular elongate member 220 may have on the tissue grasper 600. In some aspects, a bellows 340 (such as illustrated in FIG. 5) or other surface may be provided on the external elongate member 320 and/or the flexible tubular elongate member 220 to facilitate sliding of the external elongate member 320 with respect to the increased-flexibility section 222 of the flexible tubular elongate member 220. Such independent movement of the external elongate member 320 also more readily allows control of the tissue grasper 600, such as adjustment of the amount and angle of tension (e.g., magnitude and direction of the force vector) applied to tissue by the tissue grasper 600, independently of the movement of the increased-flexibility section 222 of the flexible tubular elongate member 220 of the medical scope 200. In some aspects, the tissue grasper 600 may utilize a traction device (e.g., tether, traction band, suture, string, cord, wire, filament, etc., whether elastic or inelastic) to couple the flap of tissue T to an anchoring tissue spaced apart from the treatment site S to apply traction to the tissue.

Once a procedure has been completed, the system 100 may be withdrawn from the patient. Advantageously, the return element 400 provides a homing feature returning the distal end 320d of the auxiliary device 300 to the distal end 220d of the medical scope 200 to facilitate retraction and removal of the system 100.

Although a system 100 formed in accordance with various principles of the present disclosure is illustrated in FIGS. 2-5 as allowing movement of a tissue grasper 600 or any other instrument to anchor the flap of tissue T, it will be appreciated that the auxiliary device 300 may be used to deliver other instruments independently of an instrument delivered by a medical scope 200 and without affecting the movement of the instrument delivered by the medical scope 200. Moreover, it will be appreciated that although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like. Various further benefits of the various aspects, features, components, and structures of a device and system, and associated methods, such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader’s understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system for performing a procedure within a patient’s body, said system comprising:
a first elongate member configured to deliver a medical device for use during the procedure and extending along a longitudinal axis, said first elongate member having a distal end with a distalmost end, a proximal end, and an exterior extending between said distal end and said proximal end;
a second elongate member configured to deliver a medical device for use during the procedure and extending along and coupled to said first elongate member, said second elongate member having a distal end with a distalmost end, and a proximal end, and an exterior extending between said distal end and said proximal end;
a movable member positioned between said first elongate member and said second elongate member and operable to move the distal end of said second elongate member laterally away from the distal end of said first elongate member; and
a return element coupling said first elongate member and said second elongate member, and movable to move said second elongate member laterally toward said first elongate member.

2. The system of claim 1, wherein said second elongate member is coupled to said first elongate member along the distal ends of said second elongate member and said first elongate member proximal to the distalmost ends of said second elongate member and said first elongate member.

3. The system of claim 2, further comprising an external coupler coupling the exterior of said second elongate member and the exterior of said first elongate member.

4. The system of claim 1, wherein said first elongate member defines an interior lumen therethrough, and said return element is coupled to the distal end of said second elongate member and extends proximally through the interior lumen defined through said first elongate member.

5. The system of claim 4, wherein said return element extends proximally to a control handle accessible outside the patient.

6. The system of claim 1, wherein said movable member is an expandable member.

7. The system of claim 6, wherein said expandable member extends circumferentially around the exterior of said second elongate member.

8. The system of claim 7, further comprising an inflation lumen extending along said second elongate member to be in fluid communication with said expandable member and an inflation medium source.

9. The system of claim 1, wherein said first elongate member, said second elongate member, or both of said first elongate member and said second elongate member define at least one lumen therethrough.

10. The system of claim 1, wherein:
said first elongate member is a flexible tubular elongate member of a medical scope;
said flexible tubular elongate member is configured to be inserted into the patient, defines a working channel therethrough, and has an increased-flexibility section extending from the distalmost end of said first elongate member proximally to a transition point along the distal end of said first elongate member; and
said second elongate member is coupled to said first elongate member at or along the transition point.

11. A device configured to deliver a medical instrument to a treatment site within a patient alongside another medical device, said device comprising:
an elongate member having a distal end with a distalmost end, and a proximal end, and an exterior extending between said distal end and said proximal end;
a movable member mounted on said elongate member and operable to move the distal end of said second elongate member laterally away from the other medical device; and
a return element coupled to the distal end of said elongate member and capable of being coupled to the other medical device, said return element extending proximally to a control handle accessible outside the patient and configured to move said elongate member closer to said other medical device.

12. The device of claim 11, wherein said movable member is an expandable member.

13. The device of claim 11, wherein said movable member is an inflatable member extending circumferentially around the exterior of said elongate member.

14. The device of claim 11, further comprising an external coupler configured to couple the exterior of said elongate member to the exterior of the other medical device.

15. The device of claim 11, wherein said elongate member defines a lumen extending from the proximal end of the elongate member to the distal end of the elongate member, the lumen configured for delivery of a medical instrument therethrough.

16. A method of delivering more than one medical instrument to a treatment site within a patient’s body, said method comprising:
inserting a distal end of a first elongate member and a distal end of a second elongate member into a patient's body;
advancing distalmost ends of the first elongate member and the second elongate member to the treatment site;
moving the distal end of the second elongate member laterally away from the distal end of the first elongate member; and
operating a return element to return the distal end of the second elongate member laterally toward the first elongate member.

17. The method of claim 16, further comprising coupling the distal end of the second elongate member to the distal end of the first elongate member at or proximal to a transition point between an increased flexibility section of the first elongate member extending from the distalmost end of the first elongate member proximally to the transition point and a proximally extending section of the first elongate member extending proximally from the transition point.

18. The method of claim 17, wherein the return element is coupled to the distal end of the second elongate member and extends through a working channel defined through the first elongate member, said method comprising proximally pulling on the return element to return the distal end of the second elongate member laterally toward the first elongate member.

19. The method of claim 16, further comprising expanding an expandable member to move the distal end of the second elongate member laterally away from the distal end of the first elongate member.

20. The method of claim 16, further comprising inflating an inflatable member, circumferentially extending around the exterior of the second elongate member, to move the distal end of the second elongate member laterally away from the distal end of the first elongate member.
